(19) United States
(12) Patent Application Publication
(10) Pub. No.: US 2007/0122484 A1
(43) Pub. Date: May 31, 2007

(54) Parenterally administrable microparticles

(76) Inventors: Monica Jonsson, Bara (SE); Timo Laakso, Campton (GB); Mats Reslow, Lund (SE)

Correspondence Address: MINTZ LEVIN COHN FERRIS GLOVSKY & POPEO, 666 THIRD AVENUE, NEW YORK, NY, 10017, US

(21) Appl. No.: 11/644,514

(22) Filed: Dec. 22, 2006

Related U.S. Application Data
(63) Continuation of application No. 09/970,649, filed on Oct. 5, 2001, now abandoned.
(60) Provisional application No. 60/260,496, filed on Jan. 8, 2001.

(30) Foreign Application Priority Data
Nov. 16, 2000 (SE) 0004218-4

(51) Int. Cl. A61K 31/718; A61K 9/14

(52) U.S. Cl. 424/489; 514/60

(57) ABSTRACT

A process for producing microparticles containing biologically active substance, in which process an aqueous solution of the said substance is prepared, this solution is mixed with an aqueous solution of PEG such that the substance is concentrated and/or solidified, the substance is optionally washed, the substance is mixed with an aqueous starch solution, the composition obtained is mixed, after the admixture of the starch solution, with a polymer solution, thereby forming an emulsion of starch droplets in the polymer solution, the starch droplets are solidified into microparticles, the microparticles are dried and a release-controlling shell is optionally applied to these. Novel microparticles which are obtainable by means of this process.

### TECHNICAL FIELD

The present invention lies within the field of galenic formulations for the administration of biologically active substances, more specifically microparticles for controlled release primarily intended for parenteral administration of biologically active substances, especially drugs. More specifically, it relates to a novel production process for such particles containing a biologically active substance and to novel particles for controlled release which are thereby obtainable.

### BACKGROUND TO THE INVENTION

Many drugs have to be administered by injection, since they are either subjected to degradation or are insufficiently absorbed when they are given, for example, orally or nasally or by the rectal route. A drug preparation intended for parenteral use has to meet a number of requirements in order to be approved by the regulatory authorities for use on humans. It must therefore be biocompatible and biodegradable and all used substances and their degradation products must be non-toxic. In addition, particulate drugs intended for injection have to be small enough to pass through the injection needle, which preferably means that they should be smaller than 200 μm. The drug should not be degraded in the preparation to any great extent during production or storage thereof or after administration and should be released in a biologically active form with reproducible kinetics.

One class of polymers which meets the requirements of biocompatibility and biodegradation into harmless end products is the linear polyesters based on lactic acid, glycolic acid and mixtures thereof. These polymers will also hereinafter be referred to as PLGA. PLGA is degraded by ester hydrolysis into lactic acid and glycolic acid and has been shown to possess excellent biocompatibility. The innocuous nature of PLGA can be exemplified, moreover, by the approval by the regulating authorities, including the US Food and Drug Administration, of several parenteral delayed release preparations based on these polymers.

Parenterally administrable delayed release products currently on the market and based on PLGA include Decapeptyl™ (Ibsen Biotech), Prostap SR™ (Lederle), Decapeptyl® Depot (Ferring) and Zoladex® (Zeneca). The drugs in these preparations are all peptides. In other words, they consist of amino acids condensed into a polymer having a relatively low degree of polymerization and they do not have any well-defined three-dimensional structure. This, in turn, usually allows the use of relatively stringent conditions during the production of these products. For example, extrusion and subsequent size-reduction can be utilized, which techniques would probably not be allowed in connection with proteins, since these do not, generally speaking, withstand such stringent conditions.

Consequently, there is also a need for controlled release preparations for proteins. Proteins are similar to peptides in that they also consist of amino acids, but the molecules are larger and the majority of proteins are dependent on a well-defined three-dimensional structure as regards many of their properties, including biological activity and immunogenicity. Their three-dimensional structure can be destroyed relatively easily, for example by high temperatures, surface-induced denaturation and, in many cases, exposure to organic solvents. A very serious drawback connected with the use of PLGA, which is an excellent material per se, for delayed release of proteins is therefore the need to use organic solvents to dissolve said PLGA, with the attendant risk that the stability of the protein will be compromised and that conformation changes in the protein will lead to an immunological reaction in the patient, which can produce both a loss of therapeutic effect, through the formation of inhibitory antibodies, and toxic side effects. Since it is extremely difficult to determine with certainty whether a complex protein has retained its three-dimensional structure in every respect, it is very important to avoid exposing the protein to conditions which might induce conformation changes.

Despite intense efforts aimed at modifying the PLGA technology in order to avoid this inherent problem of protein instability during the production process, progress within this field has been very slow, the main reason probably being that the three-dimensional structures for the majority of proteins are far too sensitive to withstand the manufacturing conditions used and the chemically acidic environment formed with the degradation of PLGA matrices. The scientific literature contains a large number of descriptions of stability problems in the manufacture of microspheres of PLGA owing to exposure to organic solvents. As an example of the acidic environment which is formed upon the degradation of PLGA matrices, it has recently been shown that the pH value in a PLGA microsphere having a diameter of about 40 μm falls to 1.5, which is fully sufficient to denature, or otherwise damage, many therapeutically usable proteins (Fu et al, Visual Evidence of Acidic. Environment Within Degrading Poly(lactic-co-glycolic acid) (PLGA) Microspheres, Pharmaceutical Research, Vol. 17, No. 1, 2000, 100-106). Should the microspheres have a greater diameter, the pH value can be expected to fall further owing to the fact that the acidic degradation products then get more difficult to diffuse away and the autocatalytic reaction is intensified.

The technique which is currently most commonly used to encapsulate water-soluble substances, such as proteins and peptides, is the use of multiple emulsion systems. The drug substance is dissolved in an aqueous or buffer solution and subsequently mixed with an organic solvent, immiscible with water, containing the dissolved polymer. An emulsion is formed which has the aqueous phase as the inner phase. Different types of emulsifiers and vigorous mixing are often used to create this first emulsion. This emulsion is then transferred, under is agitation, to another liquid, usually water, containing another polymer, for example polyvinyl alcohol, which produces a water/oil/water triple emulsion. The microspheres are next hardened in some way. The most common way is to utilize an organic solvent having a low boiling point, typically dichloromethane, and to distil off the solvent. If the organic solvent is not fully immiscible with water, a continuous extraction procedure can be used by adding more water to the triple emulsion. A number of variations of this general procedure are also described in the literature. In certain cases, the primary emulsion is mixed with a non-aqueous phase, for example silicone oil. Solid drug materials can also be used instead of dissolved ones.

PLGA microspheres containing proteins are described in WO-A1-9013780, in which the main feature is the use of very low temperatures during the production of the microspheres for the purpose of preserving high biological activity in the proteins. The activity for encapsulated superoxide disputation is measured, but only on the part which has been released from the particles. This method has been used to produce PLGA microspheres containing human growth hormone in WO-A1-9412158, wherein human growth hormone is dispersed in methylene chloride containing PLGA, the obtained dispersion is sprayed into a container of frozen ethanol beneath a layer of liquid nitrogen in order to freeze the fine droplets and said droplets are allowed to settle in the nitrogen on the ethanol. The ethanol is subsequently thawed and the microspheres start to sink in the ethanol, where the methylene chloride is extracted in the ethanol and the microspheres are hardened. Using this methodology, the protein stability can be better retained than in the majority of other processes for enclosing proteins in PLGA microspheres, and a product has also recently been approved by the registration authorities in the USA. However, this still remains to be clearly demonstrated for other proteins and the problem remains of exposing the enclosed biologically active substance to a very low pH during the degradation of the PLGA matrix.

In the aforementioned methods based on encapsulation with PLGA, the active substances are still exposed to an organic solvent and this, generally speaking, is harmful to the stability of a protein. Moreover, the discussed emulsion processes are complicated and probably problematical in any attempt to scale up to an industrial scale. Furthermore, many of the organic solvents which are utilized in many of these processes are associated with environmental problems and their high affinity for the PLGA polymer makes their removal difficult.

A number of attempts to solve the above-described problems caused by exposure of the biologically active substance to a chemically acidic environment during the biodegradation of the microsphere matrix and organic solvents in the manufacturing process have been described. In order to avoid an acidic environment during the degradation, attempts have been made to replace PLGA as the matrix for the microspheres by a polymer which produces chemically neutral degradation products, and in order to avoid exposing the biologically active substance to organic solvents, either it has been attempted to manufacture the microspheres in advance and, only once they have been processed and dried, to load them with the biologically active substance, or attempts have been made to exclude or limit the organic solvent during manufacture of the microspheres. A process for limiting the quantity of solvent used where polymers are used which can only be dissolved in organic solvents is described in WO 99/20253, in which the limitation is obtained by the use of an aqueous PEG solution to form an emulsion. In this publication, there is no discussion of any technique for concentrating or solidifying the biologically active substance to be incorporated in the microparticles.

By way of example, highly branched starch of relatively low molecular weight (maltodextrin, average molecular weight about 5 000 Da) has been covalently modified with acryl groups for the conversion of said starch into a form which can be solidified into microspheres and the obtained polyacryl starch has been converted into particulate form by radical polymerization in an emulsion with toluene/chloroform (4:1) as the outer phase (Characterization of Polyacryl Starch Microparticles as Carriers for Proteins and Drugs, Artursson et al, J Pharm Sci, 73, 1507-1513, 1984). Proteins were able to be entrapped in these microspheres, but the manufacturing conditions expose the biologically active substance to both organic solvents and high shearing forces in the manufacture of the emulsion. The obtained microspheres are dissolved enzymatically and the pH can be expected to be kept neutral. The obtained microspheres are not suitable for parenteral administration, especially repeated administrations, for a number of reasons. Most important of all is the incomplete and very slow biodegradability of both the starch matrix (Biodegradable Microspheres IV. Factors Affecting the Distribution and Degradation of Polyacryl Starch Microparticles, Laakso et al, J Pharm Sci 75, 962-967, 1986) and the synthetic polymer chain which cross-links the starch molecules. Moreover, these microspheres are far too small, <2 μm in diameter, to be suitable for injection in the tissues for sustained release, since tissue macrophages can easily phagocytize them. Attempts to raise the degradation rate and the degree of degradation by introducing a potentially biodegradable ester group in order to bond the acryl groups to the highly branched starch failed to produce the intended result and even these polyacryl starch microspheres were biodegraded far too slowly and incompletely over reasonable periods of time (BIODEGRADABLE MICROSPHERES: Some Properties of Polyacryl Starch Microparticles Prepared from Acrylic acid Esterified Starch, Laakso and Sjöholm, 1987 (76), pp. 935-939, J Pharm Sci.) Microspheres of polyacryl dextran have been manufactured in two-phase aqueous systems (Stenekes et al, The Preparation of Dextran Microspheres in an All-Aqueous System: Effect of the Formulation Parameters on Particle Characteristics, Pharmaceutical Research, Vol. 15, No. 4, 1998, 557-561, and Franssen and Hennink, A novel preparation method for polymeric microparticles without using organic solvents, int J Pharm 168, 1-7, 1998). With this mode of procedure, the biologically active substance is prevented from being exposed to organic solvents but, for the rest, the microspheres acquire properties equivalent to the properties described for the polyacryl starch microspheres above, which makes them unsuitable for repeated parenteral administrations. Bearing in mind that man does not have specific dextran-degrading enzymes, the degradation rate should be even lower than for polyacryl starch microspheres. The use of dextran is also associated with a certain risk of serious allergic reactions.

Manufacture of starch microspheres with the use of non-chemically-modified starch using an oil as the outer phase has been described (U.S. Pat. No. 4,713,249; Schröder, U., Crystallized carbohydrate spheres for slow release and targeting, Methods Enzymol, 1985 (112), 116-128; Schröder, U., Crystallized carbohydrate spheres as a slow release matrix for biologically active substances, Bio-materials 5:100-104, 1984). The microspheres are solidified in these cases by precipitation in acetone, which leads both to the exposure of the biologically active substance to an organic solvent and to the non-utilization, during the manufacturing process, of the natural tendency of the starch to solidify through physical cross-linking. This leads, in turn, to microspheres having inherent instability, since the starch, after resuspension in water and upon exposure to body fluids, will endeavour to form such cross-links. In order for a water-in-oil emulsion to be obtained, high shear forces are required and the microspheres which are formed are far too small to be suitable for parenteral sustained release.

EP 213303 A2 describes the production of microspheres of, inter alia, chemically unmodified starch in two-phase aqueous systems, utilizing the natural capacity of the starch to solidify through the formation of physical cross-links, and the immobilization of a substance in these microspheres for the purpose of avoiding exposure of the biologically active substance to organic solvents. The described methodology, in combination with the starch quality which is defined, does not give rise to fully biodegradable particles. Neither are the obtained particles suitable for injection, particularly for repeated injections over a longer period, since the described starch quality contains far too high quantities of foreign vegetable protein. In contrast to what is taught by this patent, it has now also surprisingly been found that significantly better yield and higher loading of the biologically active molecule can be obtained if significantly higher concentrations of the polymers are used than is required to form the two-phase aqueous system and that this also leads to advantages in terms of the conditions for obtaining stable, non-aggregated microspheres and their size distribution. The temperature treatments which are described cannot be used for sensitive macromolecules and the same applies to the processing which comprises drying with either ethanol or acetone.

Alternative methods for the manufacture of microspheres in two-phase aqueous systems have been described. In U.S. Pat. No. 5,981,719, microparticles are made by mixing the biologically active macromolecule with a polymer at a pH close to the isoelectric point of the macromolecule and stabilizing the microspheres through the supply of energy, preferably heat. The lowest percentage of macromolecule, i.e. the biologically active substance, in the preparation is 40%, which for most applications is too high and leads to great uncertainty in the injected quantity of active substance, since the dose of microparticles becomes far too low. Even though the manufacturing method is described as mild and capable of retaining the biological activity of the entrapped biologically active substance, the microparticles are stabilized by heating and, in the examples given, heating is effected to at least 58° C. for 30 min. and, in many cases, to 70-90° C. for an equivalent period, which cannot be expected to be tolerated by sensitive proteins, the biological activity of which is dependent on a three-dimensional structure, and even where the protein has apparently withstood the manufacturing process, there is still a risk of small, but nonetheless not insignificant changes in the conformation of the protein. As the outer-phase, a combination of two polymers is always used, generally polyvinyl pyrrolidone and PEG, which complicates the manufacturing process in that both these substances have to be washed off from the microspheres in a reproducible and safe way. The microparticles formed are too small (in the examples, values below 0.1 μm in diameter are quoted) to be suitable for parenteral sustained release after, for example, subcutaneous injection, since macrophages, which are cells specialized in phagocytizing particles and which are present in the tissues, are easily capable of phagocytizing microspheres up to 5-10, possibly 20 μm, and the phagocytized particles are localized intracellularly in the lysosomes, where both the particles and the biologically active substance are degraded, whereupon the therapeutic effect is lost. The very small particle size also makes the processing of the microspheres more complicated, since desirable methods, such as filtration, cannot be used. The equivalent applies to U.S. Pat. No. 5,849,864.

U.S. Pat. No. 5,578,709 and EP 0 688 429 B1 describe the use of two-phase aqueous systems for the manufacture of macromolecular microparticle solutions and chemical or thermal cross-linking of the dehydrated macromolecules to form microparticles. It is entirely undesirable to chemically cross-link the biologically active macromolecule, either with itself or with the microparticle matrix, since chemical modifications of this kind have a number of serious drawbacks, such as reduction of the bioactivity of a sensitive protein and risk of induction of an immune response to the new antigenic determinants of the protein, giving rise to the need for extensive toxicological studies to investigate the safety of the product. Microparticles which are made through chemical cross-linking with glutaraldehyde are previously known and are considered generally unsuitable for repeated administrations parenterally to humans. The microparticles which are described in U.S. Pat. No. 5,578,709 suffer in general terms from the same drawbacks as are described for U.S. Pat. No. 5,981,719, with unsuitable manufacturing conditions for sensitive proteins, either through their exposure to chemical modification or to harmful temperatures, and a microparticle size distribution which is too narrow for parenteral, sustained release and which complicates post-manufacture processing of the microspheres.

WO 97/14408 describes the use of air-suspension technology for producing microparticles for sustained release after parenteral administration, without the biologically active substance being exposed to organic solvents. However, the publication provides no guidance towards the process according to the invention or towards the novel microparticles which can thereby be obtained.

In U.S. Pat. No. 5,470,582, a microsphere consisting of PLGA and containing a macromolecule is produced by a two-stage process, in which the microsphere as such is first manufactured using organic solvents and then loaded with the macromolecule at a later stage in which the organic solvent has already been removed. This procedure leads to far too low a content of the biologically active substance, generally 1-2%, and to a very large proportion being released immediately after injection, which very often is entirely unsuitable. This far too rapid initial release is already very high given a 1% load and becomes even more pronounced when the active substance content in the microspheres is higher. Upon the degradation of the PLGA matrix, the pH falls to levels which are generally not acceptable for sensitive macromolecules.

That starch is, in theory, a very suitable, perhaps even ideal, matrix material for microparticles has been known for a long time, since starch does not need to be dissolved in organic solvents and has a natural tendency to solidify and since there are enzymes within the body which can break down the starch into endogenic and neutral substances, ultimately glucose, and since starch, presumably owing to the similarity with endogenic glycogen, has been shown to be non-immunogenic. Despite intense efforts, starch having properties which enable manufacture of microparticles suitable for parenteral use and conditions which enable manufacture of fully biodegradable microparticles under mild conditions, which allow sensitive, biologically active substances, such as proteins, to become entrapped, has not been previously described.

Starch granules naturally contain impurities, such as starch proteins, which makes them unsuitable for injection parenterally. In the event of unintentional depositing of insufficiently purified starch, such as can occur in operations where many types of operating gloves are powdered with stabilized starch granules, very serious secondary effects can arise. Neither are starch granules intrinsically suitable for repeated parenteral administrations, for the reason that they are not fully biodegradable within acceptable time spans.

Starch microspheres made of acid-hydrolyzed and purified starch have been used for parenteral administration to humans. The microspheres were made by chemical cross-linking with epichlorohydrin under strongly alkaline conditions. The chemical modification which was then acquired by the starch leads to reduced biodegradability, so that the microspheres can be fully dissolved by endogenic enzymes, such as α-amylase, but not converted fully into glucose as the end product. Neither the manufacturing method nor the obtained microspheres are suitable for the immobilization of sensitive proteins, nor is such acid-hydrolyzed starch, which is essentially based on hydrolyzed amylose, suitable for producing either fully biodegradable starch microspheres or starch microspheres containing a high load of a biologically active substance, such as a protein.

Hydroxyethyl starch (HES) is administered parenterally to humans in high doses as a plasma substitute. HES is produced by starch granules from starch consisting broadly exclusively of highly branched amylopectin, so-called “waxy maize”, being acid-hydrolyzed in order to reduce the molecular weight distribution and being subsequently hydroxyethylated under alkaline conditions and acid-hydrolyzed once more to achieve an average molecular weight of around 200,000 Da. After this, filtration, extraction with acetone and spray-drying are carried out. The purpose of the hydroxyethylation is to prolong the duration of the effect, since non-modified amylopectin is very rapidly degraded by α-amylase and its residence time in the circulation is around 10 minutes. HES is not suitable for the production of fully biodegradable microspheres containing a biologically active substance, since the chemical modification leads to a considerable fall in the speed and completeness of the biodegradation and results in the elimination of the natural tendency of the starch to solidify through the formation of non-covalent cross-linkings. Moreover, highly concentrated solutions of HES become far too viscous to be usable for the production of microparticles. The use of HES in these high doses shows that parenterally usable starch can be manufactured, even though HES is not usable for the manufacture of microspheres without chemical cross-linking or precipitation with organic solvents.

WO 99/00425 describes the use of heat-resistant proteolytic enzymes with a broad pH-optimum to purge starch granules of surface-associated proteins. The obtained granules are not suitable for parenteral administration, since they still contain the starch proteins which are present within the granules and there is a risk that residues of the added proteolytic enzymes will be left in the granules. Neither are the granules suitable for the manufacture of parenterally administrable starch microspheres in two-phase aqueous systems, since they have the wrong molecular weight distribution to be able to be used in high enough concentration, even after being dissolved, and, where microspheres can be obtained, they are probably not fully biodegradable.

The use of shearing to modify the molecular weight distribution of starch, for the purpose of producing better starch for the production of tablets, is described in U.S. Pat. No. 5,455,342 and WO 93/21008. The starch which is obtained is not suitable for parenteral administration owing to the high content of starch proteins, which might be present in denatured form after the shearing, and neither is the obtained starch suitable for producing biodegradable starch microspheres for parenteral administration or for use in two-phase aqueous systems for the production of such starch microspheres. Shearing has also been used to manufacture hydrocyethylstarch, as is disclosed in WO 96/10042. However, for similar reasons such hydrocyethylstarch is not either suitable for parenteral administration or for the production of microspheres as referred to above.

It is in many cases necessary or desirable to modify a biologically active substance, for example a drug, from soluble to solid form, for example in order to improve its stability and/or enable effective production of a formulation of the substance in question. For example, in an encapsulation procedure which utilizes an emulsifying operation, it can be necessary to use a solid form of the biologically active substance to obtain higher efficacy through the avoidance of transport to the outer phase, or the interface between the outer and inner phase, and in order to retain the biological activity of the substance. For substances which tolerate harsh manufacturing conditions, extrusion and grinding can be used, but for sensitive biologically active substances, such as proteins, it is a question in the vast majority of cases of acquiring the solid form through chemical complexing. A well known example of one such drug preparation on the market is crystalline insulin complex-bonded with zinc.

Thus it is well known that, for proteins and peptides, complex-bonding with divalent metal ions, preferably zinc, has long been utilized to convert the biologically active substance into solid form. There are however a large number of drawbacks with such procedures. One drawback is that it is not possible to form usable complexes of all interesting biologically active substances and that many complexing agents which are used in a research context are not acceptable for parenteral administration. Another drawback is the often complicated chemistry which, even in apparently simple cases, can require a significant amount of effort in order to be controlled and well-characterized. Another drawback is that the regulatory authorities in certain countries consider that even well known and marketed substances, after such complexing, are to be regarded as new chemical substances, which lead to demands for extensive and very expensive characterizations from the chemical, safety and clinical aspects. Further drawbacks are introduced when the active substance is to be converted into solid and dry form, since this often involves spraying and drying procedures which are equipment-intensive and in many cases can be complex. Many sensitive substances do not tolerate exposure to an air/water or air/organic-liquid interface or to those shearing forces which are required in order to form the spray droplets. Neither is it unusual for problems in dispersing or resuspending the substance converted into solid form, after it has been dried, not to yield a usable result, for example owing to the fact that these particles attach to one another in such a way that they cannot be driven apart by the use of acceptable conditions. In many of these procedures, organic solvents are used which risk being harmful to sensitive biologically active substances and to staff coming into contact with the substances and have an adverse effect upon the environment.

U.S. Pat. No. 5,654,010 and U.S. Pat. No. 5,664,808 describe the production of a solid form of recombinant human growth hormone, hGH, through complexing with zinc in order to create an amorphous complex, which is then micronized through an ultrasound nozzle and sprayed down in liquid nitrogen in order to freeze the droplets. The liquid nitrogen is then allowed to evaporate at a temperature of −80° C. and the resultant material is freeze-dried. Apart from the fact that the procedure is complex and generally difficult to apply, it comprises a spraying procedure in which the biologically active substance is exposed to a water/air surface and in which the amorphous form of the protein which is formed is suspended in methylene chloride. Methylene chloride is an entirely undesirable organic solvent from a toxicological viewpoint, both for the patients and for the working staff.

A process for the production of parenterally administrable microparticles and having the following features would therefore be extremely desirable:
a process which makes it possible to entrap sensitive, biologically active substances in microparticles with retention of their biological activity;
a process by means of which biologically active substances can be entrapped under conditions which do not expose them to organic solvents, high temperatures or high shear forces and which allows them to retain their biological activity;
a process which permits high loading of a parenterally administrable preparation with even sensitive, biologically active substances;
a process by means of which a substantially fully biodegradable and biocompatible preparation can be produced, which is suitable for injecting parenterally and upon whose degradation chemically neutral endogenic substances are formed;
a process by means of which a parenterally injectable preparation having a size exceeding 20 μm and, preferably exceeding 30 μm, is produced for the purpose of avoiding phagocytosis of tissue macrophages and which simplifies processing of the same during manufacture;
a process for the production of microparticles containing a biologically active substance, which microparticles can be used as intermediate product in the production of a preparation for controlled, sustained or delayed release and which permit rigorous quality control of the chemical stability and biological activity of the entrapped biological substance;
a process which utilizes a parenterally acceptable starch which is suitable for the production of substantially fully biodegradable starch microparticles;
a process which makes it possible to concentrate or solidify the biologically active substance to be incorporated in a parenterally injectable preparation, without the use of chemical completing;
a process which makes it possible to concentrate or solidify the biologically active substance to be incorporated without the use of chemical complexing and with retention of the biological activity of the substance;
a process which makes it possible to concentrate or solidify the biologically active substance to be incorporated in a parenterally administrable preparation without exposure of the substance to air/water or air/organic solvent interfaces;
a process which makes it possible to concentrate or solidify the biologically active substance to be incorporated in a parenterally administrable preparation, without the use of a spraying process or drying process;
a process which makes it possible to avoid a reconstitution stage and/or resuspension stage of the biologically active substance from dry state without prior stabilization through incorporation in microparticles;
a process which makes it possible to concentrate or solidify the biologically active substance to be incorporated without the introduction of further chemical substances, using a two-phase aqueous system for the manufacture of microparticles;
a substantially fully biodegradable and biocompatible microparticulate preparation which is suitable for injecting parenterally and upon whose degradation chemically neutral endogenic substances are formed;
a microparticulate preparation containing a biologically active substance and having a particle size distribution which is suitable for coating by means of air suspension technology and having sufficient mechanical strength for this purpose;
a coated microparticulate preparation containing a biologically active substance, which preparation gives a controlled release after parenteral administration.

### DESCRIPTION OF THE INVENTION

According to a first aspect of the present invention, there is provided a process for the production of microparticles. More specifically, it relates to the production of microparticles containing a biologically active substance and primarily intended for parenteral administration of said substance to a mammal, especially humans. Primarily, it is a question of the production of microparticles intended for injection. Since the microparticles are primarily intended for injection, it is preferably a question of the production of particles having a mean diameter within the range of 10-200 μm, usually 20-100 μm and especially 20-80 μm.

The expression “microparticles” is used in connection with the invention as a general term for particles of a certain size according to the art which is known per se. One type of microparticles is therefore constituted by microspheres, which have a substantially spherical form, whilst the term microparticle can in general include deviation from such a perfect spherical form. The term microcapsule, which is known per se, also falls within the expression microparticle according to the prior art.

More specifically the process according to the present invention comprises:
a) preparing an aqueous solution of the biologically active substance to be incorporated in the microparticles,
b) mixing the solution obtained in step a) with an aqueous solution of polyethylene glycol (PEG) under such conditions that the biologically active substance is concentrated and/or solidified,
c) optionally washing the concentrated and/or solidified biologically active substance obtained in step b),
d) mixing the concentrated and/or solidified biologically active substance obtained in step b) or c) with an aqueous starch solution,
e) mixing the composition obtained in step d) with an aqueous solution of a polymer having the ability of forming a two-phase aqueous system, so as to form an emulsion of starch droplets which contain the biologically active substance as the inner phase in an outer phase of said polymer solution,
f) causing or allowing the starch droplets obtained in step e) to solidify into starch microparticles,
g) drying the starch microparticles from step f), and
h) optionally applying a release-controlling shell of a biocompatible and biodegradable polymer to the dried starch microparticles from step f).

Even though it is generally possible to incorporate biologically active substances in microparticles in a highly effective manner, since the biologically active substance is present in soluble form during the entrapment stage, it is in certain cases preferable for the biologically active substance to be converted into solid form. For example, it can be a matter of further stabilizing the biologically active substance during the entrapment stage, of further increasing the yield or the load by converting the substance into a form which, after mixing with the inner phase (the starch solution), cannot be distributed out in the outer phase or to the interface between the inner and outer phases, or of converting the substance into a form which is as inert as possible during the manufacture of the starch microparticles, this so that improved properties shall be acquired, for example, in terms of the size distribution of the microparticles.

It has thus very surprisingly been found that PEG, which is often used as the polymer to create the outer phase in a two-phase aqueous system, can also be used to concentrate and/or solidify the biologically active substance which is to be entrapped, and that this can be realized under mild conditions which can preserve, for example, the three-dimensional conformation and biological activity of a protein.

This process has a number of advantages compared with the prior art. The basis for this is that not all biologically active substances are able to complex-bond chemically, for example with zinc, and not all complexing agents are acceptable for parenteral administration. In the first place, it is not necessary to complex-bond the biologically active substance, preferably a protein or a peptide, in order to obtain the concentration/solidification. In the second place, the use of this process often also produces better stability during the incorporation in the microparticles, compared with soluble protein. The fact that the process does not comprise a spraying or drying process before the biologically active substance is incorporated in the microparticles also means that exposure of the biologically active substance to high shearing forces and to interfaces (air/water or air/organic solvent) is avoided. Aggregation owing to electrostatic charges, something which is very common for small, dry particles, is also avoided. Any problems with wetting and resuspension of a dry powder of the biologically active substance can also be avoided. In purely general terms, spraying processes are also complex and poorly controlled. Neither is it necessary to utilize process stages such as freezing and slow thawing in order to convert the biologically active substance into dry form. It is also a distinct advantage that no organic solvents are used to convert the biologically active substance to the concentrated/solidified form.

For step a) of the process according to the invention, the aqueous solution of the biologically active substance is prepared by means of methods which are well known within the field and which do not need here to be described in further detail. However, fundamental to this is that the solution is prepared under such mild conditions, primarily in terms of temperature and agitation, that the bioactivity of the biologically active substance is preserved. Within the field, moreover, well-known buffer substances which are acceptable for parenteral use are often used to control or regulate the pH value of the solution. Where required, substances which are well known within the field and are acceptable for parenteral use can also be used, for example to adjust ionic strength and osmolarity. When so desired, the obtained solution can be sterilized by means of, for example, sterile filtration.

Through the use of the aqueous solution of polyethylene glycol in step b), a concentration of the biologically active substance, for example a protein, can be obtained. This concentration often results in the biologically active substance precipitating out, i.e. forming a precipitate, solid particles thereby being formed. This can be detected, for example, by examination with a light microscope. Since the process is often carried out quickly, the structure of the particles is generally amorphous. Other forms of particles, for example crystals and supercooled glass, are also covered by the invention, however, depending on how the process is carried out.

The term “is concentrated” also however covers the case in which the biologically active substance does not precipitate, but merely forms a more or less highly viscous solution. The term “is solidified” thus also covers the case in which a highly viscous solution of this kind forms such stable droplets that, in practice, it can be handled and incorporated in microparticles in substantially the same way as if it were a precipitation. The concentrated/solidified biologically active substance can be found in the microparticle matrix in the form of islands or discrete particles.

One embodiment of the process according to the invention is thus represented by the case in which step b) is performed such that the solidification of the biologically active substance leads to a precipitation of the same.

In another embodiment, step b) is performed such that the solidification of the biologically active substance results in a highly viscous solution, which has the ability of forming droplets which can be handled at room temperature.

In a further embodiment of the process, step b) is performed to form a reversibly solidified active substance.

In yet another embodiment of the process, the solidified biologically active substance forms a pellet or a highly viscous or solid bottom phase in centrifugation or ultracentrifugation.

By “reversibly solidified” is meant, in general terms, that the biologically active substance in question, when dissolved in a medium suitable for each unique biologically active substance and under suitable conditions, and/or when released from the microparticles in vitro and/or in vivo, is restored to essentially the same form, both chemically and biologically, as it had prior to the concentration/solidification with polyethylene glycol.

That the solidified biologically active substance forms a pellet or a highly viscous or solid bottom phase in centrifugation or ultracentrifugation provides a means of detecting the desired concentration/solidification. This means, moreover, that the substance in question is present in another physical form than the soluble form which is present in step a) after the preparation of the aqueous solution.

That the biologically active substance is present in concentrated form means, in general terms, that it is present in a concentration which exceeds the concentration obtainable when the substance in question is dissolved in an aqueous medium, with or without stabilizers and solubility-promoting substances, and with the retention of biological activity and chemical stability.

A combination of molecular weight and concentration of the PEG such that the desired concentration and/or precipitation of the biologically active substance is obtained should be chosen. Such conditions can simply be tried out for each specific biologically active substance as they will be dependent on the properties of the biologically active substance, for example molecular weight and solubility. The molecular weight of the PEG can be in the range of 400-100,000 Daltons, more preferably 4 000-35 000 Daltons, even more preferably 6 000-20,000 Daltons, and most preferably 20,000 Daltons. The concentration of the PEG can be in the range of 1-50%, preferably 2-45%, more preferably 10-40% and most preferably 20-35%. That a concentration and/or precipation has been obtained can be investigated as above. That the biologically active substance has retained its bioactivity is most easily measured at this stage by dilution, for example in a suitable buffer solution, and chemical analysis of the biologically active substance, or alternatively by suitable immunological and/or animal assays. If unsatisfactory results are obtained in the initial trials adjustment of pH, the buffer substance, or buffer substances used, and their concentration, temperature and/or inclusion of stabilizers known in the art should be investigated, as well as a change in the concentration and mean molecular weight of the PEG used, such adjustments being readily available to anyone skilled in the art. This step may obviously also be performed under an inert atmosphere to avoid oxidation reactions, the simplest way being to purge the oxygen in the solution by an inert gas, like nitrogen or helium. For extremely sensitive substances it may be necessary to use a very pure PEG to avoid, for example, oxidation reactions.

The extent to which step c) of the process according to the invention needs to be executed or not, i.e. whether the obtained concentrated and/or solidified active substance should be washed and, if so, to what extent, has to be determined in each individual case and depends, inter alia, on the proportion of the biologically active substance which is present in dissolved form in the PEG solution, on whether the dissolved substance is sufficiently stable in this form to be able to be incorporated in the microparticles without far too large a quantity of undesirable degradation products being formed, on the effect which this dissolved substance has on the manufacture of the microparticles, on whether other conditions are required to be used, for example in terms of the concentration and average molecular weight of PEG, as well as pH and ionic strength, than those employed in step b), on whether PEG constitutes a stabilizer for the biologically active substance per se or by retaining the substance in undissolved form or preventing adsorption to surfaces.

The actual washing of the concentrated and/or solidified active substance can be effected by means of suitable techniques established within this technical field. In the simplest form of all, centrifugation washes can be used and in many cases filtration can also be used. In the latter case, conditions are preferably employed under which the concentrated and/or solidified active substance is not allowed to dry, since this can lead, for example, to aggregation, and the process time is shortened by the application of pressure. Fundamental to this, of course, is that the liquid which is used should not dissolve the concentrated and/or solidified active substance and that the conditions which are suitable should be determined for each individual biologically active substance. In many cases, conditions can be chosen, in terms of buffer composition, additives and temperatures, such that this requirement is met, and necessary information can be obtained from the literature or via simple experiments. Naturally, polymers can be added to avoid dissolution of the concentrated and/or solidified active substance and, in the simplest case of all, the same composition of the PEG solution is used as when the concentration/solidification was carried out.

A starch which is especially suitable in connection with the process according to the invention, as well as a process for the production thereof, is accurately described in the Swedish patent application No. 0003616-0. Another suitable starch is disclosed in a copending PCT application having the same filing date as the present application and entitled STARCH. Details relating to the starches and their production can be obtained, in other words, from said patent applications, the contents of which in this regard are thus herewith included by reference in the present text.

The most important features of such a starch will, however, be described below. In order that fully biodegradable microparticles with high active substance yield shall be formed in a two-phase aqueous system and in order that the obtained starch microparticles shall have the properties to be described below, the starch must generally predominantly consist of highly branched starch, which, in the natural state in the starch granule, is referred to as amylopectin. It should also have a molecular weight distribution which makes it possible to achieve desired concentrations and gelation rates. In the two cases referred to in the previous paragraph said molecular weight distribution can be accomplished by means of shearing or acid hydrolysis, respectively.

It may be added, in this context, that the term “biodegradable” means that the microparticles, after parenteral administration, are dissolved in the body to form endogenic substances, ultimately, for example, glucose. The biodegradability can be determined or examined through incubation with a suitable enzyme, for example alpha-amylase, in vitro. It is in this case appropriate to add the enzyme a number of times during the incubation period, so as thereby to ensure that there is active enzyme permanently present in the incubation mixture. The biodegradability can also be examined through parenteral injection of the microparticles, for example subcutaneously or intramuscularly, and histological examination of the tissue as a function of time.

Normally, biodegradable starch microparticles disappear from the tissue within a few weeks and often within one week. In those cases in which the starch microparticles are coated with a release-controlling shell, for example by the application of a thin layer, it is generally this shell which determines the biodegradability rate, which then, in turn, determines when alpha-amylase becomes available to the starch matrix.

The biocompatibility can also be examined through parenteral administration of the microparticles, for example subcutaneously or intramuscularly, and histological evaluation of the tissue, it being important to bear in mind that the biologically active substance, which often is a protein, has in itself the capacity to induce, for example, an immune response if administered to another species. For example, a large number of recombinantly produced human proteins can give rise to an immune response in test animals.

The starch must further have a purity which is acceptable for the manufacture of a parenterally administrable preparation. It must also be able to form sufficiently stable solutions in sufficiently high concentration to enable the biologically active substance to be mixed in under conditions allowing the retention of the bioactivity of the substance, at the same time as it must spontaneously be able to be solidified in a controlled manner in order to achieve stable, yet at the same time biodegradable, microparticles. High concentration of the starch is also important to prevent the biologically active substance from being distributed out to an unacceptable extent to the outer phase or to the interface between the inner and the outer phases.

A number of preferred embodiments with regard to the nature of the starch are as follows.

The starch preferably has an amylopectin content exceeding 85% by weight, the molecular weight of said amylopectin being reduced so that at least 80% by weight of the material lies within the range of 10-10 000 kDa.

In addition, the starch preferably has an amino acid nitrogen content of less than 50 μg per g dry weight of starch.

The starch preferably has a purity of at most 20 μg, more preferably at most 10 μg, and most preferably at most 5 μg, amino acid nitrogen per g dry weight of starch.

The molecular weight of the abovementioned amylopectin is preferably reduced, for instance by shearing, by acid hydrolysis or by enzymatic hydrolysis, for example with isoamylase, such that at least 80% by weight of the material lies within the range of 100-4 000 kDa, more preferably 200-1 000 kDa, and most preferably 300-600 kDa.

In addition, the starch preferably has an amylopectin content with the reduced molecular weight in question exceeding 95% by weight, more preferably exceeding 98% by weight. It can also, of course, consist of 100% by weight of such amylopectin.

According to another preferred embodiment, the starch is of such a type that it can be dissolved in water in a concentration exceeding 25% by weight. This means, in general, a capacity to dissolve in water according to a technique which is known per se, i.e. usually dissolution at elevated temperature, for example up to approximately 80° C.

According to a further preferred embodiment, the starch is substantially lacking in covalently bonded extra chemical groups of the type which are found in hydroxyethyl starch. By this is meant, in general, that the starch essentially only contains groups of the type which are found in natural starch and have not been in any way modified, such as in hydroxyethyl starch, for example.

Another preferred embodiment involves the starch having an endotoxin content of less than 25 EU/g.

A further preferred embodiment involves the starch containing less than 100 microorganisms per g, often even leas than 10 microorganisms per g.

The starch can further be defined as being substantially purified from surface-located proteins, lipids and endotoxins by means of washing with an aqueous alkali solution, reduced in molecular weight by means of shearing or acid hydrolysis and purified from internal proteins by means of ion-exchange chromatography, preferably anion-exchange chromatography, or gel electrophoresis.

As far as the purity in all these contexts is concerned, it is in general the case that expressions of the type “essentially” or “substantially” generally mean at least of 90%, for example 95%, 99% or 99.9%.

That amylopectin constitutes the main component part of the starch used means in general terms that its percentage is 60-100% by weight, calculated on the basis of dry weight of starch.

In certain cases, it can here be favourable to use a lesser percentage, for example 2-15% by weight, of short-chain amylose to modify the gelation rate in step f). The average molecular weight of the said amylose lies preferably within the range of 2.5-70 kDa, especially 5-45 kDa. Other details regarding short-chain amylose can be obtained from U.S. Pat. No. 3,881,991.

In the formation of the starch solution which is used in step d), heating according to a technique which is known per se is in general used to dissolve the starch. An especially preferred embodiment simultaneously involves the starch being dissolved under autoclaving, it also preferably being sterilized. This autoclaving is realized in aqueous solutions, for example water for injection or suitable buffer.

If the biologically active substance is a sensitive protein or another temperature-sensitive substance, the starch solution will have to cool to an appropriate temperature before being combined with the substance in question. What temperature is appropriate is determined firstly by the thermal stability of the biologically active substance, but in purely general terms a temperature of less than ca. 60° C., preferably less than 55° C., is appropriate.

According to a preferred embodiment, the active substance(s) is/are therefore combined with the starch solution at a temperature of at most 60° C., more preferably at most 55° C., and preferably within the range of 20-45° C., especially 30-37° C.

For the mixing operation in step d), furthermore, a weight ratio of starch:biologically active substance within the range of 3:1 to 10 000:1 is expediently used.

As has been discussed above, it is also the case for the mixing operation that the active substance is concentrated/solidified with the use of a PEG solution before being mixed with the starch solution. It is possible to add the starch solution to the biologically active substance or vice versa. After this, a homogeneous distribution of the concentrated/solidified active substance in the starch solution is created by means of a suitable technique. Such a technique is well known within the field, examples which might be quoted being magnetic agitation, propeller agitation or the use of one or more static mixers.

In the production of the starch microparticles according to the present invention, the concentration of starch in the solution which is to be converted to solid form and in which the biologically active substance is to be incorporated should be at least 20% by weight to enable the formation of starch microparticles having good properties. Exactly what starch concentration works best in each individual case can be titrated out in a simple manner for each individual biologically active substance, where the load in the microparticles is that which is required in the individual case. In this context, it should be noted that the biologically active substance to be incorporated in the microparticles can affect the two-phase system and the gelation properties of the starch, which also means that customary preparatory trials are conducted for the purpose of determining the optimal conditions in the individual case. Trials generally show that the starch concentration should advantageously be at least 30% by weight and in certain specific cases at least 40% by weight. As the highest limit, 50% by weight is usually applicable, especially at most 45% by weight. It is not normally possible to obtain these high starch concentrations without the use of molecular-weight-reduced, highly branched starches.

Regarding the polymer used in step e) for the purpose of forming a two-phase aqueous system, information is published, within precisely this technical field, on a large number of polymers with the capacity to form two-phase systems with starch as the inner phase. All such polymers must be considered to lie within the scope of the present invention. An especially suitable polymer in this context, however, is polyethylene glycol. This polyethylene glycol preferably has an average molecular weight of 5-35 kDa, more preferably 15-25 kDa and especially about 20 kDa.

The polymer is dissolved in suitable concentration in water or aqueous solution, which expression also includes buffer solution, and is temperature-adjusted to a suitable temperature. This temperature lies preferably within the range of 4-50° C., more preferably 10-40° C. and often 10-37° C. The concentration of the polymer in the water-based solution is expediently at least 20% by weight and preferably at least 30% by weight, and expediently at most 45% by weight. An especially preferred range is 30-40% by weight.

The mixing operation in step e) can be performed in many different ways, for example through the use of propeller agitation or at least one static mixer. The mixing is normally carried out within the temperature range of 4-50° C., preferably 20-40° C., often about 37° C. In a batch process, the starch solution can be added to the polymer solution or vice versa. Where static mixers or blenders are utilized, the operation is expediently executed by the two solutions being pumped in two separate pipelines into a common pipeline containing the blenders.

The emulsion can be formed using low shearing forces, since there is no high surface tension present between the phases in water/water emulsions, in contrast to oil/water or water/oil emulsions, and in this case it is primarily the viscosity of the starch solution which has to be overcome for the droplets to achieve a certain size distribution. In most cases, magnetic or propeller agitation is sufficient. On a larger scale, for example when the quantity of microparticles to be produced exceeds 50 g, it is expedient to use so-called baffles to obtain even more effective agitation in the container which is used. An alternative way of forming the water/water emulsion is to use at least one static mixer, the starch solution expediently being pumped at regulated speed in a pipe in which the static mixers have been placed. The pumping can be effected with any type of suitable pump, provided that it gives an even flow rate under these conditions, does not expose the mixture to unnecessarily high shearing forces and is acceptable for the manufacture of parenteral preparations in terms of purity and non-leakage of unwanted substances. In those cases, too, in which static mixers are used to create the emulsion, it is generally advantageous to have the solidification into microparticles take place in a vessel with suitable agitation.

A preferred embodiment of the process according to the invention means that in step e) the polymer solution is added to the composition in at least two stages, in which an addition is effected after the emulsion has been created or has begun to be created.

It is also within the scope of the present invention, of course, to add the polymer solutions in many stages and to change, for example, the average molecular weight and/or concentration of the polymer used, for example in order to increase the starch concentration in the inner phase where this is desirable.

The mixing operation in step e) is expediently performed, moreover, under such conditions that the formed starch droplets acquire the size required for the microparticles, i.e. preferably a mean diameter, in the dry state, within the range of 10-200 μm, more preferably 20-100 μm and most preferably 20-80 μm.

In connection with the solidification of the microparticles, it is important that this should take place under conditions which are mild for the incorporated biologically active substance(s) In other words, it is primarily a question of using a temperature which is not harmful to the current substance In this context, it has surprisingly been shown that the criteria for this and for the formation of stable microparticles with suitable size distribution can more easily be met if, during the solidification, more than one temperature or temperature level is used. It is especially advantageous if the solidification process in the two-phase system is initiated at a lower temperature than the temperature which is used in the end phase of the solidification. A preferred embodiment means that the solidification is initiated within the range of 1-20° C., preferably 1-10° C., especially around 4° C., and is terminated within the range of 20-55° C., preferably 25-40° C., especially around 37° C.

Confirmation that the chosen conditions are correct or appropriate can be obtained by establishing that the starch microparticles have a desired size distribution, are stable during the subsequent washing and drying operations and are dissolved substantially by fully enzymatic means in vitro and/or that the incorporated substance has been encapsulated effectively and has retained bioactivity. The last-mentioned is usually examined using chromatographic methods or using other methods established within the art, in vitro or in vivo, after the microparticles have been enzymatically dissolved under mild conditions, and is an important element in ensuring a robust and reliable manufacturing process for sensitive, biologically active substances. It is a great advantage for the microparticles to be able to be fully dissolved under mild conditions, since this minimizes the risks of preparation-induced artifacts, which are usually found when, for example, organic solvents are required to dissolve the microparticles, which is the case, for example, when these consist of a PLGA matrix.

The formed microparticles are preferably washed in a suitable manner in order to remove the outer phase and any surplus of active substance. Such washing is expediently effected by filtration, which is made possible by the good mechanical stability and suitable size distribution of the microparticles. Washing by means of centrifugation, removal of the supernatant and resuspension in the washing medium may often also be appropriate. In each washing process, one or more suitable washing media are used, which generally are buffer-containing aqueous solutions. In this connection, sifting can also be used, if required, in order to adjust the size distribution of the microparticles, for example to eliminate the content of too small microparticles and to ensure that no microparticles above a certain size are present in the finished product.

The microparticles can be dried in any way appropriate, for example by spray-drying, freeze-drying or vacuum-drying. Which drying method is chosen in the individual case often depends on what is most appropriate for the retention of the biological activity for the enclosed biologically active substance. Process considerations also enter into the picture, such as capacity and purity aspects. Freeze-drying is often the preferred drying method, since, correctly designed, it is especially mild with respect to the enclosed biologically active substance. That the incorporated biologically active substance has retained its bioactivity can be established by means of analysis appropriate to the substance after the microparticle has been enzymatically dissolved under mild conditions. Suitable enzymes for use in connection with starch are alpha-amylase and amyloglucosidase, singly or in combination, it being important to establish, where appropriate, that they are free from possible proteases, which can degrade proteins. The presence of proteases can be detected with methods known within the field and, for example, by mixing the biologically active substance in control trials and determining its integrity in the usual manner after incubation with the intended enzyme mixture under the conditions which will afterwards be used to dissolve the microparticles. Where the preparation is found to contain protease contamination, it can be replaced by a preparation which offers higher purity or is purged of proteases. This can be done using techniques known within the field, for example by chromatography with α₂-macroglobulin bonded to suitable chromatographic material.

In order to modify the release properties of the microparticles, a release-controlling shell made from a biocompatible and biodegradable polymer might also be applied, moreover. Examples of suitable polymers in this context are found in the prior art, and polymers of lactic acid and glycolic acid (PLGA) can especially be mentioned. The shell in question is preferably applied using air suspension technology. An especially suitable technique of this kind is described in WO97/14408 and details in this regard can thus be obtained from this publication, the content of which is included in the text by reference. The starch microparticles which are obtained by means of the process according to the present invention are extremely well suited to be coated, e.g. by the application of a thin layer, by means of the said air suspension technology, and the coated microparticles obtained are especially well suited to parenteral administration.

When the produced microparticles are used, either they are coated with a release-controlling outer shell or not, and the dry microparticles are suspended in a suitable medium, specifically to permit injection. Such media and processes in these regards are well known within the field and will not need here to be described in further detail. The actual injection can be given through a suitable needle or with a needle-free injector. It is also possible to inject the microparticles using a dry powder injector, without prior resuspension in an injection medium.

Apart from the advantages which have been discussed above, the process according to the invention has the advantage that the yield of the biologically active substance is generally high, that it is possible to obtain a very high active substance content in the microparticles whilst retaining the bioactivity of the substance, that the obtained microparticles have the correct size distribution for use for parenteral, controlled (for example delayed or sustained) release, since they are too large to be phagocytized by macrophages and small enough to be injectable through small needles, for example 23G-25G, and that endogenic and neutral degradation products are formed upon degradation of the microparticles, by which means the active substance, for example, can be prevented from being exposed to an excessively low pH value. Moreover, the process itself is especially well suited to rigorous quality control.

The process according to the invention is especially interesting in connection with proteins, peptides, polypeptides, polynucleotides and polysaccharides or, in general, other drugs or biologically active substances which are sensitive to or unstable in, for example, organic solvents. Recombinantly produced proteins are a very interesting group of biologically active substances. Generally speaking, however, the invention is not limited to the presence of such substances, since the inventive concept is applicable to any biologically active substance which can be used for parenteral administration. Apart from in connection with sensitivity or instability problems, the invention can thus also be of special interest in such cases where it would otherwise be difficult to remove solvent or where toxicological or other environmental problems might arise.

Examples of biologically active substances of the above-specified type are growth hormone, erythropoietin, interferon (α, β, γ-type), vaccine, epidermal growth hormone, Factors IV, V, VI, VII, VIII and IX, LHRH-analogue, insulin, macrophage-colony-stimulating factor, granulocyte-colony-stimulating factor and interleukin.

Usable biologically active substances of the non-protein drug type can be chosen from the following groups:

Antitumour agents, antibiotics, anti-inflammatory agents, antihistamines, sedatives, muscle-relaxants, antiepileptic agents, antidepressants, antiallergic agents, bronchodilators, cardiotonic agents, antiarrhythmic agents, vasodilators, antidiabetics, anticoagulants, haemostatic agents, narcotics and steroids.

According to another aspect of the invention, this also relates to novel microparticles of the type which can be produced by means of the process according to the invention. The novel microparticles according to the invention are not limited, however, to those which can be produced by means of the said process, but comprise all microparticles of the type in question irrespective of the production methods.

More precisely, it is a question of microparticles suitable for parenteral administration, preferably via injection, to a mammal, especially a human being, and containing a biologically active substance, which microparticles essentially have the same properties as the microparticles obtainable by means of the process described above.

According to one aspect of the invention, these are represented by microparticles essentially consisting of parenterally administrable, biodegradable starch as the matrix, which contains the biologically active substance in essentially non-chemically complex-bonded form and in the form of solid particles having a mean size within the range of 0.05-30 μm.

By mean size is usually meant, in this context, mean diameter, at least in the case of spherical or substantially spherical particles. In another configuration, reference is generally to the mean value for the largest extent of the particle in any direction.

According to one embodiment of the invention, the particles of the biologically active substance are obtained by precipitation, i.e. are present in precipitated form.

The solid particles preferably have a mean size within the range of 0.2-10 μm, more preferrably 0.5-5 μm, and most preferably 1-4 μm.

Another embodiment is represented by microparticles in which the starch has an amino acid nitrogen content of less than 50 μg per g dry weight of starch and which microparticles have no covalent chemical cross-linking between the starch molecules.

Another embodiment relates to microparticles in which the starch has an amylopectin content exceeding 85 percent by weight, of which at least 80 percent by weight has a average molecular weight within the range of 10-1 000 kDa.

The starch can otherwise have the features which have been discussed in connection with the process.

Preferably, the microparticles also have a release-controlling shell of the type discussed in connection with the process. Reference is also made to the process regarding preferred variants of the said shell.

Other microparticles according to the invention are those in which the bioactivity of the biologically active substance is at least 80%, preferably at least 90% and most preferably essentially maintained compared with the bioactivity exhibited by the substance prior to its incorporation in the starch.

Other microparticles according to the invention are those which are biodegradable in vitro in the presence of alpha-amylase and/or amyloglucosidase.

Others still are those which are biodegradable and are eliminated from tissue after subcutaneous or intramuscular administration. The biologically active substance is preferably a protein and more preferably a recombinantly produced protein.

The protein is preferably chosen from amongst protein hormones, preferably growth hormones, coagulation factors, preferably FVII, VII, VIII and IX, LHRH analogues, insulin and insulin analogues, C-peptide, glucagon-like peptides, LHRH analogues, leptines, colony-stimulating factors, preferably macrophage-colony-stimulating factor, granulocyte-stimulating factor and granulocyte/macrophage-stimulating factor, interferons, preferably interferon α, interferon β and interferon γ, interleukins, and recombinantly produced vaccines.

More preferably the protein is a growth hormone, especially a human growth hormone (hGH).

That the biologically active substance in the starch matrix is present in essentially non-chemically complex-bonded form means in general that the molecular ratio of total metal cations:biologically active substance is less than 0.2:1.

According to the prior art, it is primarily zinc which has been utilized for complex-bonding in similar context. Thus, the microparticles according to the invention have the advantage that they are essentially or wholly lacking in such zinc.

More preferably, the abovementioned molecular ratio of metal cations:biologically active substance is less than 0.1, especially less than 0.01:1, and most preferably, of course, as close to 0 as possible.

Where a human growth hormone constitutes the biologically active substance, this is preferably of the type whose dimers content is less than 2% by weight, and more preferably less than 1% by weight, and whose polymers content is less than 0.2% by weight, preferably less than 0.1% by weight.

A further preferred embodiment of the microparticles according to the invention is constituted by those in which the biologically active substance is human growth hormone and for which the release kinetics for the said hGH determined in vitro are characterized by substantially continuous and regular release over at least one week.

Microparticles which form a parenterally administrable, biodegradable microparticle preparation containing a biologically active substance which, during the first 24 hours after injection, has an active substance release which is less than 30% of the total release, determined from a concentration-time graph in the form of the ratio between area under the curve during the first 24 hours and total area under the curve in question.

Preferably, the release during the first 24 hours after the injection is less than 20%, more preferably less than 15%, even more preferably less than 10% and most preferably less than 5%, of the total release.

Microparticles which produce a microparticle preparation of the abovementioned type, which, during the first 48 hours after injection, has an active substance release in which the maximum concentration in plasma or serum is less than 300% of the maximum concentration of the biologically active substance during any point in time in excess of 48 hours after injection.

The said maximum concentration is preferably less than 200% and more preferably less than 100% of the maximum concentration in question.

Another example is a microparticle preparation of the abovementioned type, which has a biologically active substance release in which the bioavailability of the said substance is at least 35% of the bioavailability obtained when the substance in question is injected intravenously in soluble form.

The said bioavailability is preferably at least 45%, more preferably at least 50%, of the bioavailability obtained when the biologically active substance is injected intravenously.

A further example is a microparticle preparation of the said type which has an active substance release characterized in that, in the release occurring during any continuous seven-day period, the quotient of the highest concentration of the biologically active substance in serum or plasma divided by the mean concentration during the said seven-day period is less than 5, provided that the chosen seven-day period does not include the first 24 hours after injection.

The said release is preferably less than 4 times, more preferably less than 3 times, and most preferably less than 2 times.

Another microparticle preparation which can be obtained by means of the microparticles according to the invention has a biologically active substance release in which the mean residence time for the substance in question is at least 4 days.

Preferably, the said mean residence time is at least 7 days, more preferably at least 9 days, for example at least 11 days, or especially at least 13 days.

The features which have been specified for the above-presented microparticle preparations can be combined in any suitable combinations whatsoever.

The different characteristics specified for the microparticle preparation above primarily relate to the terms MRT, burst and bioavailability.

These can be defined as follows:

MRT

An object of preparations for controlled release is to obtain a sustained release of the active material. One measure which can be used to quantify the release time is mean residence time (MRT), which is the recognized term within pharmacokinetics.

MRT is the average time for which the molecules introduced into the body reside within the body. (Clinical Pharmacokinetics. Concepts and Applications. Malcolm Rowland and Thomas N. Tozer, 2^{nd} ed., Lea&Febiger, Philadelphia London).

The MRT value can be calculated from plasma concentration data, using the following formula.

$$MRT = \frac{{\int }_{0}^{∞} tC ⅆ t}{{\int }_{0}^{∞} C ⅆ t}$$

where C is the plasma concentration and t is the time.
Burst

A common problem with controlled release preparations for parenteral use is that a large part of the drug is released during the early phase immediately following administration in the body. Within the specialist literature, this is termed the “burst effect”. This is generally due to the fact that the drug is located on the surface of the formulation or that the formulation (which can consist of microparticles) bursts. A low burst effect is very desirable, since a high concentration of drug can be toxic and the part which disappears rapidly in the initial period, moreover, is poorly utilized, which means that more drug is required to maintain a therapeutic level of the drug during the intended treatment period.

Burst is defined as that fraction of the drug which is absorbed during the first 24 hours of the total fraction which is absorbed.

In mathematical terms, it can be defined using “area under curve” calculations from plasma concentration graphs.

$$Burst = \frac{{\int }_{0}^{24 h} C ⅆ t}{{\int }_{0}^{∞} C ⅆ t} \cdot 100 \%$$

Bioavailability

Bioavailability is a measure of how large a part of the supplied drug is absorbed in active form from the site of administration to the blood. Bioavailability is often compared with data from intravenous supply of the drug, in which there are therefore no absorption barriers, and is then, referred to as absolute bioavailability.

Absolute bioavailability is defined according to the following formula:

$$F = \frac{{AUC}_{x} \cdot {D}_{iv}}{{D}_{x} \cdot {AUC}_{iv}}$$

where AUCₓ is the area-under-the-curve value for the examined formulation, AUCᵢᵥ is the area-under-the-curve value for an intravenous supply of the drug, Dₓ is the dose of the drug in the formulation and Dᵢᵥ is the intravenous dose.

The determination of the release profile and the pharmacokinetic parameters is preferably realized through animal trials. The most relevant species, owing to its similarity to humans, is the pig. Where the biologically active substance can induce, during the test, an immune response which threatens to affect the determination of the pharmacokinetic parameters for the biologically active substance, inhibition of the immune response should be used, for example by drug treatment. This is known within the technical field, and details can be obtained from the scientific literature, for example Agersõ et al, (J. Pharmacol Toxicol 41 (1999) 1-8).

Other interesting microparticles according to the invention are those which are biodegradable in vitro in the presence of alpha-amylase and/or amyloglucosidase.

Further preferred microparticles are those which are biodegradable and are eliminated from tissue after subcutaneous or intramuscular administration.

As regards the determination of the biological activity of the microparticles containing active substance, this must be carried out in a manner appropriate to each individual biological substance. Where the determination is effected in the form of animal trials, a certain quantity of the biologically active substance incorporated in the starch microparticles is injected, possibly after these microparticles have been previously enzymatically dissolved under mild conditions, and the biological response is compared with the response obtained after injection of a corresponding quantity of the same biologically active substance in a suitable solution. Where the evaluation is made in vitro, for example in test tubes or in cell culture, the biologically active substance is preferably made fully available before the evaluation by the starch microparticles being enzymatically dissolved under mild conditions, after which the activity is determined and compared with the activity for a control solution having the same concentration of the biologically active substance in question. In any event, the evaluation shall include any non-specific effects of the degradation products of the starch microparticles.

### EXAMPLES

The invention will now be further illustrated by the non-limiting illustrative embodiments below.

### Example 1

Procedure for the Production of Highly Concentrated/Precipitated hGH Suitable for Immobilization with PEG.

To 343 mg hGH are added 10 mM sodium phosphate buffer, pH 6.4, to a total volume of 2.5 ml. PEG with a average molecular weight of 20,000 D is dissolved in the same buffer to a concentration of 30%, the pH being adjusted to about 6.4. The PEG solution (25 ml) is poured into a beaker having a propeller, after which the temperature is adjusted to 15° C. and the hGH solution (about 1.25 ml) is added under propeller agitation and the mixture allowed to stand for 75 min. under continued agitation. The obtained suspension is centrifuged in a Sorvall SS34 (20 min. at 5 000 rpm). The supernatant is carefully drawn off. The precipitated protein can be washed once with sodium acetate, pH 6.4, containing 2 mM zinc acetate (10 ml) and the obtained supernatant is drawn off.

### Example 2

Procedure for the Immobilization of PEG-Solidified hGH in Starch Microspheres Made from Highly Branched Sheared Starch.

Starch microspheres are made from sheared starch with an average molecular weight of 390 kDa. The starch is dissolved by heating in 10 mM sodium phosphate, pH 6.4, to a concentration of 40% and the obtained starch solution is allowed to cool to about 55° C. After this, 2.1 g of the obtained starch solution are mixed with the whole of the batch of hGH manufactured in Example 1, suspended in 10 mM sodium acetate buffer containing 2 mM zinc acetate, pH 6.4, total 2.9 ml, the mixture being agitated until a homogeneous suspension of the protein in the starch solution is formed. To the obtained suspension are added 12 g of a PEG solution of 42% concentration, in which the average molecular weight of PEG is 20 kDa. The solidification is initiated at 4° C. for 17 hours and concluded at 37° C. for 6 hours. The obtained starch microspheres containing hGH are washed three times with 38 ml 10 mM sodium acetate buffer containing 2 mM zinc acetate, pH 6.4, and freeze-dried. The obtained microspheres are dissolved with α-amylase and the quantity of incorporated hGH is determined, for example by means of analysis with high-pressure-liquid chromatography. The fraction of dimer and polymer is also determined, for example using high-pressure-liquid chromatography. The yield of starch microspheres containing hGH is generally at least 80% and the hGH content, expressed as dry weight, is around 15 percent by weight. The dimer content of the protein is generally <1% and the polymer content <0.1%, which shows that the protein is acceptable for parenteral administration to humans.

### Example 3

Procedure for Coating of Starch Microspheres Containing PEG-Concentrated hGH.

The hGH-containing starch microspheres obtained in Example 2 are coated with a release-controlling shell made from PLGA by means of air suspension technology according to WO97/14408 with the use of a mixture consisting of 75% RG502H and 25% RG756 (both from Boehringer Ingelheim). After the coating operation, the coating is dissolved with a mixture of methylene chloride and acetone in a ratio of 1:3 and, after these solvents have been washed away, for example by repeated centrifugation, the microspheres are dissolved with α-amylase. The hGH content is determined, for example by analysis with high-pressure-liquid chromatography. The dimer and polymer contents of the protein are also determined using the same technique. The protein content can be around 11 percent by weight. The fraction of the protein which is present in the form of dimers is <2% and in the form of polymers <0.1%. The release kinetics for hGH from the coated microspheres can be determined in vitro and are characterized by the absence of an undesirable burst and otherwise by a continuous and regular release with a duration of around one week. With this process, parenterally administrable microspheres can thus be produced so as to be suitable for controlled release of hGH.

### Example 4

Procedure for the Production of Highly Concentrated/Precipitated hGH Suitable for Immobilization With the use of PEG.

Precipitated hGH is produced according to Example 1, with the change that the precipitate is washed in histidine buffer, pH 4.9.

What is claimed:

1. A process for producing parenterally administrable microparticles containing a biologically active substance, which process comprises:
a) preparing an aqueous solution of the biologically active substance to be incorporated in the microparticles;
b) mixing the solution obtained in step a) with an aqueous solution of polyethylene glycol to form a concentrated biologically active substance, said concentrated biologically active substance selected from a concentrated solution of the biologically active substance or a solid particle precipitate of the biologically active substance;
c) optionally washing the concentrated biologically active substance obtained in step b);
d) mixing the concentrated biologically active substance obtained in step b) or step c) with an aqueous starch solution to form a composition comprising the concentrated biologically active substance and the starch solution;
e) mixing the composition obtained in step d) with an aqueous solution of a polymer having the ability to form a two-phase aqueous system comprising an emulsion of starch droplets which contain the biologically active substance as the inner phase in an outer phase of the polymer solution;
f) causing or allowing the starch droplets obtained in step e) to solidify into starch microparticles;
g) drying the solid starch microparticles from step f); and
h) coating the dried solid starch microparticles from step g) with a release controlling shell of a biocompatible and biodegradable polymer.

2. The process according to claim 1, in which step b) is performed such that the biologically active substance precipitates from the aqueous solution obtained in step a) to form solid particles.

3. The process according to claim 1, in which step b) is performed such that a concentrated solution of the biologically active substance is formed, wherein the concentrated solution has a viscosity that differs from the viscosity of the solution obtained step a) and the ability to form droplets which can be handled at room temperature.

4. The process according to claim 1, wherein the biologically active substance of step b) is restored to essentially the same chemical and biological form as said biologically active substance prior to concentration or precipitation.

5. The process according to claim 1, in which the concentrated solution of biologically active substance forms a pellet or a solution of higher viscosity than the concentrated solution or solid bottom phase in centrifugation or ultracentrifugation.

6. The process according to claim 1, in which the polyethylene glycol used in step b) has an average molecular weight of 400 to 100,000 Da.

7. The process according to claim 1, in which the concentration of the polyethylene glycol used in step b) is in the range of 1-50% (w/w).

8. The process according to claim 1, in which, in step d), an aqueous starch solution is utilized, comprising starch which has an amylopectin content exceeding 85% by weight, in which the molecular weight of the said amylopectin has been reduced such that at least 80% by weight of the material lies within the range of 10-10,000 kDa.

9. The process according to claim 1, in which, in step d), an aqueous starch solution is utilized, comprising starch which has an amino acid nitrogen content of less than 50 μg per gram dry weight of starch.

10. The process according to claim 1, in which the starch concentration of the aqueous starch solution used in step d) is at least 20% by weight.

11. The process according to claim 8, in which the starch has a purity of at most 20 μg amino acid nitrogen per gram dry weight of starch.

12. The process according to claim 8, in which the starch has an amylopectin content with said reduced molecular weight exceeding 95% by weight.

13. The process according to claim 8, in which the molecular weight of said amylopectin has been reduced such that at least 80% by weight of the material lies within the range of 100-4000 kDa.

14. The process according to claim 1, in which the starch is such that it can only be dissolved to a concentration exceeding 25% by weight in water.

15. The process according to claim 1, in which the starch lacks covalently bonded extra chemical groups which are found in hydroxyethyl starch.

16. The process according to claim 1, in which the starch has an endotoxin content of less than 25 EU/g and contains less than 100 microorganisms per gram.

17. The process according to claim 1, in which the starch is essentially purified from surface-located proteins, lipids and endotoxins by means of washing with an aqueous alkali solution and purified from internal proteins by means of ion-exchange chromatography.

18. The process according to claim 8, in which, in step d), 215% by weight amylose is also used as starch, having an average molecular weight within the range of 2.5-70 kDa in which the percentage by weight is calculated on the basis of dry weight starch.

19. The process according to claim 8, in which, in step d), a solution is prepared having a starch concentration of at least 30% by weight.

20. The process according to claim 8, in which, in step d), a solution is prepared having a starch concentration of at most 50% by weight.

21. The process according to claim 8, in which the aqueous starch solution in step d) is prepared with accompanying autoclaving.

22. The process according to claim 1, in which, in step d), the active substance is combined with the starch solution at a temperature of at most 60° C.

23. The process according to claim 1, in which, in step d), the weight ratio between starch and biologically active substance in the composition lies within the range of 3:1 to 10000:1.

24. The process according to claim 1, in which the mixing in step e) is performed at a temperature within the range of 4-50° C.

25. The process according to claim 1, in which the mixing in step e) is performed by means of at least one static mixer.

26. The process according to claim 1, in which, in step e), the polymer solution is added to the composition in at least two steps, at least one of the addition steps being effected after the emulsion has begun to be created.

27. The process according to claim 1, in which, in step e), polyethylene glycol is used as the aqueous polymer.

28. The process according to claim 27, in which the polyethylene glycol has an average molecular weight of 5-35 kDa.

29. The process according to claim 1, in which, in step e), starch droplets are formed which yield microparticles comprising a mean particle diameter size within the range of 10-200 μm, in the dry state.

30. The process according to claim 29, in which, after step e), the microparticles are washed, through filtration, and optionally sieved in order to obtain the desired particle size distribution.

31. The process according to claim 1, in which the solidification in step f) is effected at least two temperatures, in which initiation of solidification is effected at a lower temperature than termination of solidification.

32. The process according to claim 31, in which the solidification in step f) is initiated within the range of 1-20° C., and is terminated within the range of 20 to 55° C.

33. The process according to claim 1, in which the drying in step g) is performed in the form of spray-drying, freeze-drying, or vacuum-drying.

34. The process according to claim 1, in which the biologically active substance comprising the microparticles comprises one or more of proteins, peptides, polypeptides, polynucleotides, or polysaccharides.

35. The process according to claim 1, in which the application of the release-controlling shell in step h) is performed by means of air suspension technology.

36. The process according to claim 1, in which the release-controlling shell in step h) comprises a homopolymer or copolymer containing alpha-hydroxy acid units.

37. The process according to claim 36, in which the alpha-hydroxy acid is lactic acid and/or glycolic acid.

38. The process according to claim 7, in which the concentration of the polyethylene glycol used in step b) is in the range of 2-45% (w/w).

39. The process according to claim 7, in which the concentration of the polyethylene glycol used in step b) is in the range of 10-40% (w/w).

40. The process according to claim 7, in which the concentration of the polyethylene glycol used in step b) is in the range of 20-30% (w/w).

41. The process according to claim 11, in which the starch has a purity of at most 10 μg amino acid nitrogen per gram dry weight of starch.

42. The process according to claim 11, in which the starch has a purity of at most 5 μg amino acid nitrogen per gram dry weight of starch.

43. The process according to claim 12, in which the starch has an amylopectin content with said reduced molecular weight exceeding 98% by weight.

44. The process according to claim 13, in which the molecular weight of said amylopectin has been reduced such that at least 80% by weight of the amylopectin lies within the range of 200-1000 kDa.

45. The process according to claim 13, in which the molecular weight of said amylopectin has been reduced such that at least 80% by weight of the amylopectin lies within the range of 300-600 kDa.

46. The process according to claim 18, in which in step d)₁ 215% by weight amylose is also used as starch, having an average molecular weight within the range of 5-45 kDa in which the percentage by weight is calculated on the basis of dry weight starch.

47. The process according to claim 20, in which, in step d), a solution is prepared having a starch concentration of at most 45% by weight.

48. The process according to claim 22, in which, in step d), the active substance is combined with the starch solution at a temperature of 20-45° C.

49. The process according to claim 22, in which, in step d), the active substance is combined with the starch solution at a temperature of 30-37° C.

50. The process according to claim 24, in which the mixing in step e) is performed at a temperature within the range of 10-40° C.

51. The process according to claim 24, in which the mixing in step e) is performed at a temperature within the range of 10-37° C.

52. The process according to claim 28, in which the polyethylene glycol has an average molecular weight of 15-25 kDa.

53. The process according to claim 28, in which the polyethylene glycol has an average molecular weight of about 20 kDa.

54. The process according to claim 29, in which, in step e), starch droplets are formed which yield microparticles comprising a size within the range of 20-100 μm, in the dry state.

55. The process according to claim 29, in which, in step e), starch droplets are formed which yield microparticles comprising a size within the range of 20-80 μm, in the dry state.

56. The process according to claim 32, in which the solidification is initiated within the range of 1-10° C., and is terminated within the range of 20-55° C.

57. The process according to claim 32, in which the solidification is initiated around 4° C., and is terminated within the range of 20-55° C.

58. The process according to claim 32, in which the solidification is initiated within the range of 1-20° C., and is terminated within the range of 20-40° C.

59. The process according to claim 32, in which the solidification is initiated within the range of 1-20° C., and is terminated around 37° C.

60. The process according to claim 33, in which the drying in step g) is performed by freeze-drying.

61. The process according to claim 34, wherein the protein is a recombinantly produced protein.

62. The process according to claim 1, in which the polyethylene glycol used in step b) has an average molecular weight of 4,000 to 35,000 Da.

63. The process according to claim 1, in which the polyethylene glycol used in step b) has an average molecular weight of 6,000 to 20,000 Da.

64. The process according to claim 1, in which the polyethylene glycol used in step b) has an average molecular weight of about 20,000 Da.

65. The process according to claim 17, in which the ion-exchange chromatography is anion exchange chromatography.

66. The process according to claim 34, wherein the protein is human growth hormone (hGH).

67. The process according to claim 1, wherein, in step (h), the biocompatible and biodegradable polymer comprises polymers of lactic acid and glycolic acid (PLGA).

68. The process according to claim 1, wherein, in step (h), the biocompatible and biodegradable polymer is applied as a coating by air suspension technology.

69. The process according to claim 1, wherein, in step (h), the biocompatible and biodegradable polymer is applied in the presence of an organic solvent.

70. A process for producing parenterally administrable microparticles containing a biologically active substance, which process comprises:
a) preparing an aqueous solution of the biologically active substance to be incorporated in the microparticles;
b) mixing the solution obtained in step a) with an aqueous solution of polyethylene glycol to form a concentrated biologically active substance, said concentrated biologically active substance selected from a concentrated solution of the biologically active substance or a solid particle precipitate of the biologically active substance;
c) optionally washing the concentrated biologically active substance obtained in step b);
d) mixing the concentrated biologically active substance obtained in step b) or step c) with an aqueous starch solution to form a composition comprising the concentrated biologically active substance and the starch solution;
e) mixing the composition obtained in step d) with an aqueous solution of a polymer having the ability to form a two-phase aqueous system comprising an emulsion of starch droplets which contain the biologically active substance as the inner phase in an outer phase of the polymer solution;
f) causing or allowing the starch droplets obtained in step e) to solidify into starch microparticles; wherein solidification is effected at least two temperatures, with initiation of solidification being at a lower temperature than termination of solidification;
g) drying the solid starch microparticles from step f); and
h) coating the dried solid starch microparticles from step g) with a release controlling shell of a biocompatible and biodegradable polymer.

71. A process for producing parenterally administrable microparticles containing a biologically active substance, which process comprises:
a) preparing an aqueous solution of the biologically active substance to be incorporated in the microparticles;
b) mixing the solution obtained in step a) with an aqueous solution of polyethylene glycol to form a concentrated biologically active substance, said concentrated biologically active substance selected from a concentrated solution of the biologically active substance or a solid particle precipitate of the biologically active substance;
c) optionally washing the concentrated biologically active substance obtained in step b);
d) mixing the concentrated biologically active substance obtained in step b) or step c) with an aqueous starch solution to form a composition comprising the concentrated biologically active substance and the starch solution;
e) mixing the composition obtained in step d) with an aqueous solution of a polymer having the ability to form a two-phase aqueous system comprising an emulsion of starch droplets which contain the biologically active substance as the inner phase in an outer phase of the polymer solution;
f) causing or allowing the starch droplets obtained in step e) to solidify into starch microparticles;
g) drying the solid starch microparticles from step f); and
h) coating the dried solid starch microparticles from step g) by air suspension technology with a release controlling shell comprising biocompatible and biodegradable polymer of lactic acid and glycolic acid (PLGA).

72. The process according to claim 70, wherein, in step (h), the biocompatible and biodegradable polymer of lactic acid and glycolic acid (PLGA) is applied in the presence of an organic solvent.

73. The process according to claim 71 wherein, in step (h), the biocompatible and biodegradable polymer of lactic acid and glycolic acid (PLGA) is applied in the presence of an organic solvent.

74. The process according to claim 70, in which the solidification in step f) is initiated within the range of 1-20° C., and is terminated within the range of 20 to 55° C.

75. The process according to claim 70, in which the solidification is initiated within the range of 1-10° C., and is terminated within the range of 20-55° C.

76. The process according to claim 70, in which the solidification is initiated within the range of 1-20° C., and is terminated within the range of 20-40° C.

77. The process according to claim 70, in which the solidification is initiated within the range of 1-20° C., and is terminated around 37° C.

78. The process according to claim 70, in which the solidification is initiated around 4° C., and is terminated within the range of 20-55° C.

79. The process according to claim 70, in which the drying in step g) is performed in the form of spray-drying, freeze-drying, or vacuum-drying.

80. The process according to claim 70, in which the biologically active substance comprising the microparticles comprises one or more of proteins, peptides, polypeptides, polynucleotides, or polysaccharides.

81. The process according to claim 70, wherein the protein is a recombinantly produced protein.

82. The process according to claim 70, in which the application of the release-controlling shell in step h) is performed by means of air suspension technology.

83. The process according to claim 70, in which the release-controlling shell in step h) comprises a homopolymer or copolymer containing alpha-hydroxy acid units.

84. The process according to claim 70, in which the alpha-hydroxy acid is lactic acid and/or glycolic acid.

85. The process according to claim 70, in which the concentration of the polyethylene glycol used in step b) is in the range of 1-50% (w/w).

86. The process according to claim 70, in which the molecular weight of said amylopectin has been reduced such that at least 80% by weight of the amylopectin lies within the range of 200-1000 kDa.

87. The process according to claim 70, in which, in step d), the active substance is combined with the starch solution at a temperature of 20-45° C.

88. The process according to claim 70, in which the mixing in step e) is performed at a temperature within the range of 10-40° C.

89. The process according to claim 70, in which the polyethylene glycol has an average molecular weight of 15-25 kDa.

90. The process according to claim 70, in which, in step e), starch droplets are formed which yield microparticles comprising a size within the range of 20-100 μm, in the dry state.
